(19) United States
(12) Patent Application Publication
(10) Pub. No.: US 2022/0415433 A1
(43) Pub. Date: Dec. 29, 2022

(54) DRUG SCREENING METHOD AND APPARATUS, AND ELECTRONIC DEVICE

(71) Applicant: Tencent Technology (Shenzhen) Company Limited, Shenzhen (CN)

(72) Inventors: Tingyang XU, Shenzhen (CN); Jiying ZHANG, Shenzhen (CN); Fei YE, Shenzhen (CN); Yu RONG, Shenzhen (CN); Junzhou HUANG, Shenzhen (CN)

(73) Assignee: Tencent Technology (Shenzhen) Company Limited, Shenzhen (CN)

(21) Appl. No.: 17/900,149

(22) Filed: Aug. 31, 2022

Related U.S. Application Data
(63) Continuation of application No. PCT/CN2021/107509, filed on Jul. 21, 2021.

(30) Foreign Application Priority Data
Jul. 21, 2020 (CN) 202010704024.0

(51) Int. Cl. G16B 15/30; G16C 20/50; G16B 40/20

(52) U.S. Cl. CPC G16B 15/30; G16C 20/50; G16B 40/20

(57) ABSTRACT

This disclosure provides a drug screening method and apparatus, an electronic device, and a computer-readable storage medium. The method includes: determining a structural feature of a protein molecule and a structural feature of a target molecule; obtaining a concatenated node feature corresponding to the protein molecule and the target molecule based on a node information passing sub-network in a drug screening model, the structural feature of the protein molecule, and the structural feature of the target molecule, the node information passing sub-network being a graph neural network; and predicting a first predicted activity value after the protein molecule and the target molecule are bound according to the concatenated node feature.

### RELATED APPLICATION

This application is a continuation of International Application No. PCT/CN2021/107509, filed Jul. 21, 2021, which claims priority to Chinese Patent Application No. 202010704024.0 filed on Jul. 21, 2020, both of which are incorporated herein by reference in their entirety.

### TECHNICAL FIELD

This application relates to information processing technologies, and in particular, to a drug screening method and apparatus, an electronic device, and a computer-readable storage medium.

### BACKGROUND

Drug research and development is a high-tech industry that has received extensive attention in modern society. Drug screening is a key link in drug research and development, which refers to the assessment of activity or other properties of substances (such as proteins) that may be used as drugs.

However, in a conventional drug screening process, research and development personnel usually conduct relevant experiments manually for drug screening, which results in high cost, long research and development cycle, and low success rate in drug screening. Moreover, existing systems and processes are ineffective in successfully assessing activities or other properties of substances and determining which substances can be used as drugs.

There is no effective solution to this problem in the related art.

### SUMMARY

Embodiments of this disclosure provide a drug screening method, performed by an electronic device, the method including:

obtaining a protein molecule and a target molecule included in a drug database;

determining a structural feature of the protein molecule and a structural feature of the target molecule;

obtain a concatenated node feature corresponding to the protein molecule and the target molecule based on a node information passing sub-network in a drug screening model, the structural feature of the protein molecule, and the structural feature of the target molecule, the node information passing sub-network being a graph neural network (GNN); and

predicting a first predicted activity value after the protein molecule and the target molecule are bound according to the concatenated node feature.

The embodiments of this disclosure further provide a drug screening apparatus, including:

an information transmission module, configured to obtain a protein molecule and a target molecule included in a drug database; and

an information processing module, configured to:

determine a structural feature of the protein molecule and a structural feature of the target molecule;

obtain a concatenated node feature corresponding to the protein molecule and the target molecule based on a node information passing sub-network in a drug screening model, the structural feature of the protein molecule, and the structural feature of the target molecule, the node information passing sub-network being a graph neural network (GNN); and

predict a first predicted activity value after the protein molecule and the target molecule are bound according to the concatenated node feature.

The embodiments of this disclosure further provide an electronic device, including:

a memory, configured to store an executable instruction; and

a processor, configured to implement, when executing the executable instruction stored in the memory, the drug screening method provided in the embodiments of this disclosure.

The embodiments of this disclosure further provide a non-transitory computer-readable storage medium, storing an executable instruction, the executable instruction, when executed by a processor, implementing the drug screening method provided in the embodiments of this disclosure.

### BRIEF DESCRIPTION OF THE DRAWINGS

FIG. 1 is a schematic diagram of an application scenario of a drug screening method according to an embodiment of this disclosure.

FIG. 2 is a schematic diagram of a composition structure of an electronic device according to an embodiment of this disclosure.

FIG. 3A is a schematic flowchart of a drug screening method according to an embodiment of this disclosure.

FIG. 3B is a schematic flowchart of a drug screening method according to an embodiment of this disclosure.

FIG. 4 is a schematic diagram of determining a structural feature of a protein molecule according to an embodiment of this disclosure.

FIG. 5 is a schematic flowchart of determining a structural feature of a target molecule according to an embodiment of this disclosure.

FIG. 6 is a schematic diagram of determining a structural feature of a target molecule according to an embodiment of this disclosure.

FIG. 7 is a schematic flowchart of a drug screening method according to an embodiment of this disclosure.

FIG. 8 is a schematic diagram of a processing process of training a drug screening model according to an embodiment of this disclosure.

### DESCRIPTION OF EMBODIMENTS

To make objectives, technical solutions, and advantages of this disclosure clearer, the following further describes this disclosure in detail with reference to the accompanying drawings. The described embodiments are not to be considered as a limitation to this disclosure. All other embodiments obtained by a person of ordinary skill in the art without creative efforts shall fall within the protection scope of this disclosure.

In the following descriptions, the term “some embodiments” describes subsets of all possible embodiments, but it may be understood that “some embodiments” may be the same subset or different subsets of all the possible embodiments, and can be combined with each other without conflict.

In the following descriptions, the involved term “first/second” is merely intended to distinguish similar objects but does not necessarily indicate a specific order of objects. It may be understood that “first/second” is interchangeable in terms of a specific order or sequence if permitted, so that the embodiments of this disclosure described herein can be implemented in a sequence in addition to the sequence shown or described herein. In the following descriptions, the term “plurality of” means at least two.

Before the embodiments of this disclosure are further described in detail, a description is made on terms in the embodiments of this disclosure, and the terms in the embodiments of this disclosure are applicable to the following explanations.

(1) “In response to” is used for representing a condition or status on which one or more operations to be performed depend. When the condition or status is satisfied, the one or more operations may be performed immediately or after a set delay. Unless explicitly stated, there is no limitation on the order in which the plurality of operations are performed.

(2) “Based on” is used for representing a condition or status on which one or more operations to be performed depend. When the condition or status is satisfied, the one or more operations may be performed immediately or after a set delay. Unless explicitly stated, there is no limitation on the order in which the plurality of operations are performed.

(3) “Molecule” is a whole composed of atoms bound with each other according to a certain bonding sequence and spatial arrangement. The relationship between the bonding sequence and spatial arrangement is referred to as a molecular structure. In the embodiments of this disclosure, a macromolecule may refer to a biological substance with a relative molecular mass of 5000 or more, such as protein, nucleic acid, and polysaccharide; and a micro-molecule may refer to a biological substance with a relative molecular mass of 1000 or less, such as peptide, oligopeptide, oligosaccharide, oligonucleotide, and vitamin.

(4) “Protein molecule” is a substance with a certain spatial structure formed by coiling and folding of a polypeptide chain composed of amino acids in the manner of “dehydration condensation”.

(5) In the embodiments of this disclosure, “drug screening” refers to simulating a process of drug screening on a computer to predict a possible activity of a compound, so as to perform targeted entity screening on compounds that are more likely to become drugs. It can be expressed as screening molecular structures that need to know a drug target by using a molecular docking technology, calculating the capability of binding a micro-molecule to the target in a compound library through molecular simulation, and predicting a physiological activity of a candidate compound. The keys to drug screening include establishing a proper pharmacophore model, accurately determining or predicting a molecular structure of a target protein, and accurately and quickly calculating free energy change of the interaction between a candidate compound and a target.

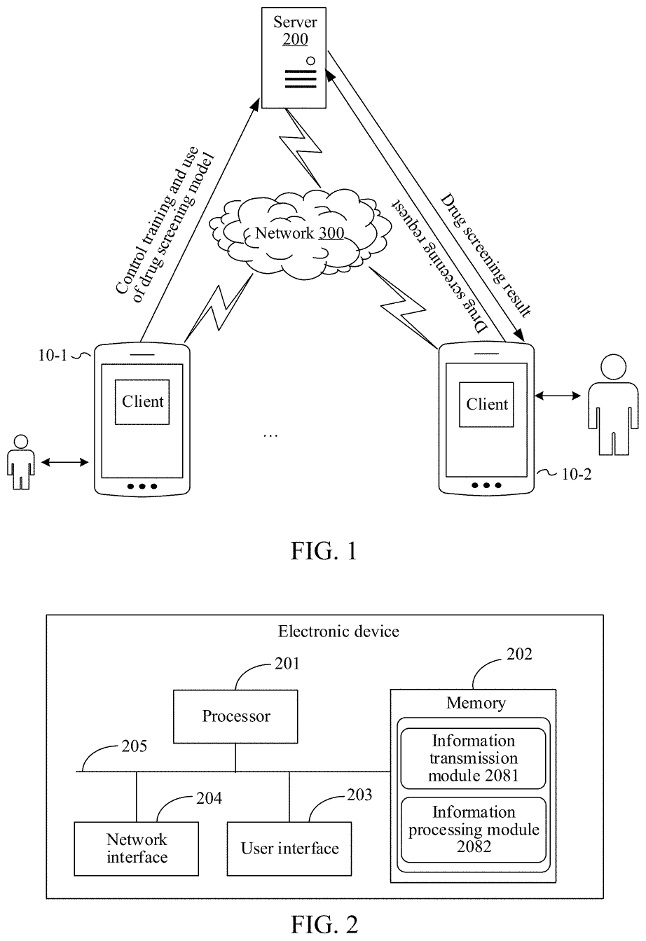

FIG. 1 is a schematic diagram of an application scenario of a drug screening method according to an embodiment of this disclosure. Referring to FIG. 1, a terminal includes a terminal 10-1 and a terminal 10-2. The terminal 10-1 is located at a developer side to control training and use of a drug screening model. The terminal 10-2 is located at a user side to request drug screening. The terminal is connected to a server 200 through a network 300. The network 300 may be a wide area network, a local area network, or a combination of the wide area network and the local area network, and implements data transmission by using a wireless or wired link.

The terminal 10-2 is located at a user side to transmit a request for drug screening to request screening of protein molecules and target molecules included in a drug database.

In some embodiments, the server 200 is configured to arrange a drug screening apparatus to implement the drug screening method provided in this disclosure. The server 200 may arrange a trained drug screening model to implement drug screening in different environments (for example, an environment of screening targeted drugs or chemical drugs). Before a drug screening model is used, the drug screening model may be trained. An exemplary process includes: determining a training sample set matching the drug screening model based on drug information parameters in a drug database, the training sample set including at least one group of training samples; extracting a feature set matching the training samples through the drug screening model; and training the drug screening model according to the feature set matching the training samples to determine model parameters fitting the drug screening model. Certainly, the drug screening apparatus provided in this disclosure may train drug screening models corresponding to the same target molecule in different drug screening environments, and finally display activity detection results (such as a first predicted activity value and/or a second predicted activity value) of a binding product of a protein molecule and a target molecule determined through the drug screening model on a user interface (UI). The activity detection results may also be called by other application programs. Certainly, a drug screening model matching a corresponding drug database may alternatively be transferred to different drug screening processes (such as a targeted drug screening process, a chemical drug screening process, or a polymer drug screening process).

After the drug screening model is trained, the server 200 may perform drug screening through the drug screening model. An exemplary process includes: obtaining a protein molecule and a target molecule included in a drug database; determining a structural feature of a protein molecule and a structural feature of a target molecule; obtaining a concatenated node feature corresponding to the protein molecule and the target molecule based on a node information passing sub-network in a drug screening model, the structural feature of the protein molecule, and the structural feature of the target molecule, the node information passing sub-network being a graph neural network (GNN); and predicting a first predicted activity value after the protein molecule and the target molecule are bound according to the concatenated node feature. The server 200 may screen molecules in the drug database based on the first predicted activity value obtained for drug screening. After the drug screening is completed, screening results may be transmitted to the terminal such as the terminal 10-1 or the terminal 10-2. The drug screening may also be performed in combination with a second predicted activity value. A process of determining the second predicted activity value will be described later.

In some embodiments, the terminal (such as the terminal 10-1 or the terminal 10-2) may arrange the drug screening apparatus to implement the drug screening method provided in this disclosure. That is, the drug screening model is trained locally on the terminal, and the drug screening is performed according to the trained drug screening model.

In some embodiments, the server 200 may train the drug screening model and transmit the trained drug screening model to the terminal, so that the terminal implements drug screening locally according to the trained drug screening model.

The following describes a structure of an electronic device according to an embodiment of this disclosure in detail. The electronic device may be implemented in various forms, such as a dedicated terminal with a drug screening apparatus processing function, or a server with a drug screening apparatus processing function, for example, the server 200 in FIG. 1. FIG. 2 is a schematic diagram of a composition structure of an electronic device according to an embodiment of this disclosure. It may be understood that, FIG. 2 shows only an exemplary structure rather than a complete structure of the electronic device. The structure shown in FIG. 2 may be partially or entirely implemented as required.

The electronic device provided in this embodiment of this disclosure includes: at least one processor 201, a memory 202, a user interface 203, and at least one network interface 204. The components in the electronic device are coupled by using a bus system 205. It may be understood that the bus system 205 is configured to implement connection and communication between the components. In addition to a data bus, the bus system 205 further includes a power bus, a control bus, and a status signal bus. However, for ease of clear description, all types of buses are marked as the bus system 205 in FIG. 2.

The user interface 203 may include a display, a keyboard, a mouse, a track ball, a click wheel, a key, a button, a touch panel, a touchscreen, or the like.

It may be understood that, the memory 202 may be a volatile memory or a non-volatile (non-transitory) memory, or may include both a volatile memory and a non-volatile memory. The memory 202 in this embodiment of this disclosure can store data to support operation of the terminal (such as the terminal 10-1 or the terminal 10-2 shown in FIG. 1). An example of the data includes any computer program to be operated on the terminal (such as the terminal 10-1 or the terminal 10-2 shown in FIG. 1), for example, an operating system and an application program. The operating system includes various system programs, such as framework layers, kernel library layers, and driver layers used for implementing various basic services and processing hardware-based tasks. The application program may include various application programs.

In some embodiments, a drug screening apparatus provided in the embodiments of this disclosure may be implemented in the form of a combination of software and hardware. In an example, the drug screening apparatus provided in the embodiments of this disclosure may be a processor in the form of a hardware decoding processor, and is programmed to execute the drug screening method provided in the embodiments of this disclosure. For example, the processor in the form of the hardware decoding processor may use one or more application specific integrated circuits (ASICs), digital signal processors (DSPs), programmable logic devices (PLDs), complex programmable logic devices (CPLDs), field-programmable gate arrays (FPGAs), or other electronic elements.

In an example in which the drug screening apparatus provided in the embodiments of this disclosure is implemented by a combination of software and hardware, the drug screening apparatus provided in the embodiments of this disclosure may be directly embodied as a combination of software modules executed by the processor 201. The software modules may be located in a storage medium (a computer-readable storage medium), and the storage medium is located in the memory 202. The processor 201 reads executable instructions included in the software modules in the memory 202 and uses necessary hardware (for example, including the processor 201 and other components connected to the bus system 205) in combination, to implement the drug screening method provided in the embodiments of this disclosure.

For example, the processor 201 may be an integrated circuit chip and has a signal processing capability, such as a general purpose processor, a digital signal processor (DSP), or another programmable logical device, a discrete gate, a transistor logical device, or a discrete hardware component. The general purpose processor may be a microprocessor or any conventional processor.

In an example in which the drug screening apparatus provided in the embodiments of this disclosure is implemented by using hardware, the apparatus provided in the embodiments of this disclosure may be implemented directly by using the processor 201 in the form of a hardware decoding processor, for example, the drug screening method provided in the embodiments of this disclosure is performed by one or more ASICs, DSPs, PLDs, CPLDs, FPGAs, or other electronic elements.

The memory 202 in this embodiment of this disclosure is configured to store various types of data to support operation of the drug screening apparatus. An example of the data includes: any executable instruction to be operated on the drug screening apparatus. A program that implements the drug screening method of the embodiments of this disclosure may be included in the executable instruction.

In some embodiments, the drug screening apparatus provided in the embodiments of this disclosure may be implemented in the form of software. FIG. 2 shows the drug screening apparatus stored in the memory 202, which may be software in the form of a program, a plug-in, or the like, and include a series of modules. An example of the program stored in the memory 202 may include the following software modules: an information transmission module 2081 and an information processing module 2082. When the software modules in the drug screening apparatus are read by the processor 201 into a random access memory (RAM) and executed, the drug screening method provided in the embodiments of this disclosure will be implemented.

In some embodiments, the server 200 may be an independent physical server, or may be a server cluster or a distributed system formed by a plurality of physical servers, or may be a cloud server that provides basic cloud computing services such as a cloud service, a cloud database, cloud computing, a cloud function, cloud storage, a network service, cloud communication, a middleware service, a domain name service, a security service, a content delivery network (CDN), big data, and an artificial intelligence platform. The server 200 may be a physical device or a virtualized device. The terminal (such as the terminal 10-1 or the terminal 10-2 shown in FIG. 1) may be a smartphone, a tablet computer, a notebook computer, a desktop computer, or the like, which is not limited herein. The terminal and the server may be directly or indirectly connected in a wired or wireless communication manner. This is not limited in the embodiments of this disclosure.

During actual application, the drug screening model provided in the embodiments of this disclosure may be applied to the fields of structural biology and medicine, and drug discovery, molecular optimization, molecular synthesis, and the like can be achieved through the drug screening model.

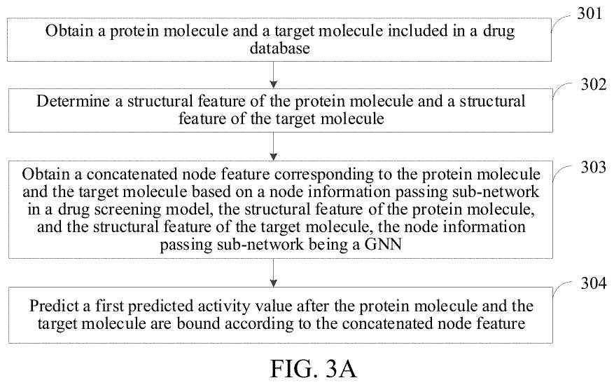

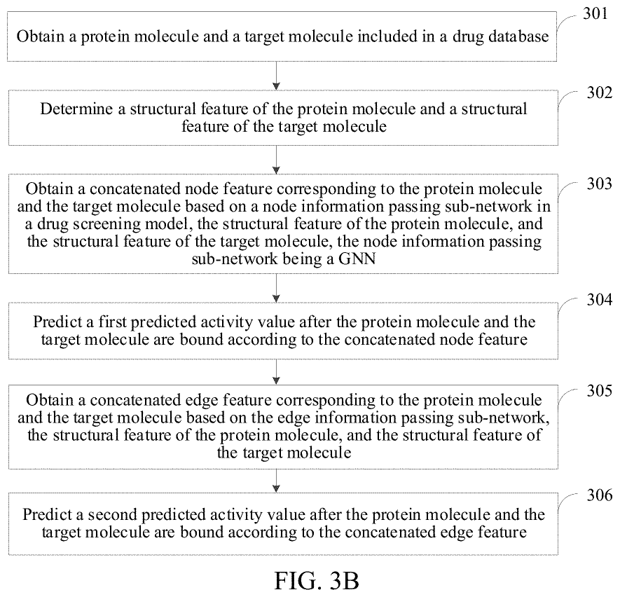

The drug screening method provided in the embodiments of this disclosure is described with reference to the drug screening apparatus shown in FIG. 2. FIG. 3A is a schematic flowchart of a drug screening method according to an embodiment of this disclosure. It may be understood that, steps shown in FIG. 3A may be performed by various electronic devices running the drug screening apparatus, such as a dedicated terminal with a drug screening apparatus, a drug database server, or a server cluster for drug providers.

In FIG. 3A, in order to overcome the defects of low accuracy and low efficiency in drug screening caused by a conventional drug screening method, the technical solutions provided by this disclosure adopt artificial intelligence technology, thereby greatly reducing the time and cost required for related experiments, while increasing the accuracy of drug screening and improving the efficiency of drug screening. The following specifically describes steps shown in FIG. 3A.

Step 301: Obtain a protein molecule and a target molecule included in a drug database.

For example, when receiving a drug screening request (from a user or another electronic device), an electronic device obtains a protein molecule and a target molecule included in a drug database. The target molecule may be a drug micro-molecule, and the protein molecule may be a target macromolecule that can be acted on by a drug molecule (for example, a drug micro-molecule).

If a possible activity of a compound in the drug database is required to be predicted to perform targeted entity screening on compounds that may become clinical drugs, a molecular docking technology can be used for implementation. For example, the target macromolecule that can be acted on by a drug molecule and the drug micro-molecule are concatenated to form a new compound, and a physiological activity of the formed compound is predicted.

Step 302: Determine a structural feature of the protein molecule and a structural feature of the target molecule.

In some embodiments of this disclosure, the determining a structural feature of the protein molecule and a structural feature of the target molecule may be implemented in the following manner:

determining spatial positions of different amino acid chains in the protein molecule; obtaining a normalized amino acid distance by determining a distance between amino acids in each pair based on the spatial positions of different amino acid chains, and normalizing the distance between amino acids in each pair; determining an amino acid matrix diagram corresponding to the protein molecule based on the normalized amino acid distance and an amino acid distance threshold; determining the structural feature of the protein molecule based on the amino acid matrix diagram corresponding to the protein molecule; and determining atoms and chemical bonds corresponding to the target molecule, and determining the structural feature of the target molecule based on the atoms and the chemical bonds corresponding to the target molecule.

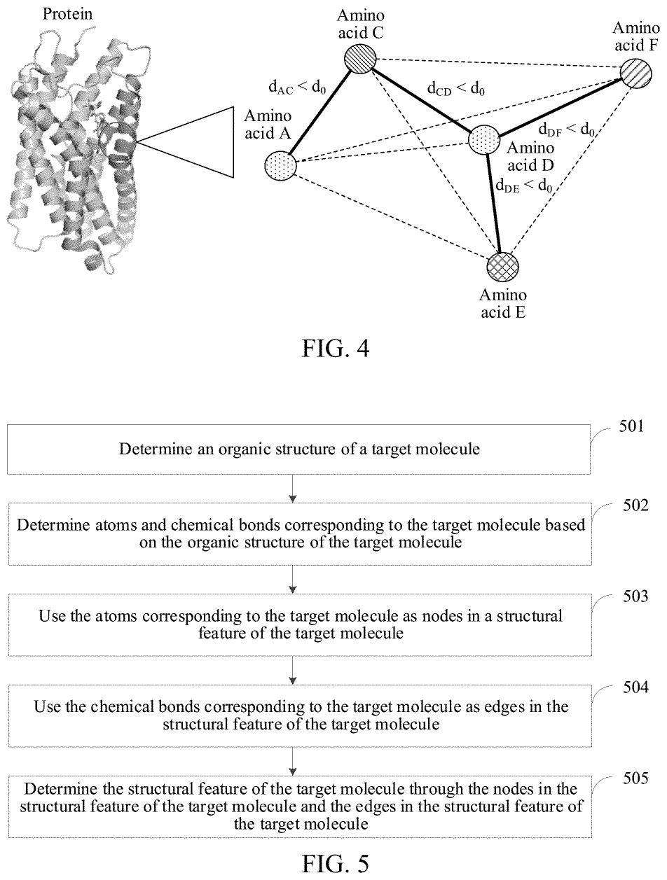

For example, FIG. 4 is a schematic structural diagram of a protein molecule in an embodiment of this disclosure. During drug screening, since a structure of a molecule cannot be directly inputted into a neural network for training and learning, it may be projected into a vectorized space, that is, it may be characterized. In terms of molecular representation, since a molecule is formed by establishing connections among different atoms by chemical bonds, the molecule may be regarded as a graph composed of nodes and edges. For example, referring to FIG. 4, a protein has a spatial structure and is formed by folding of amino acid chains in the spatial structure, and a distance between amino acids in each pair can be calculated based on its spatial structure. A normalized spatial distance between amino acids is calculated according to the following formula 1:

$${\overline{S}}_{ij} = \frac{1}{1 + {d}_{ij} / d ’} \qquad Formula 1$$

d^{·} is a scale used for normalization. For example, d^{·} may be 3.8 ^{·}A. S̅ᵢⱼ represents a distance from an i^{th} amino acid to a j^{th} amino acid. After the normalized amino acid distance (such as dᵢⱼ) is obtained, an adjacency matrix (that is, an amino acid matrix diagram) of a protein graph can be calculated with reference to a fixed threshold d₀ (that is, an amino acid distance threshold). The adjacency matrix of the protein graph is calculated according to the formula 2:

$${A}_{ij} = \{ \begin{matrix}1 , {d}_{ij} < {d}_{0} \\ 0 , {d}_{ij} > {d}_{0}\end{matrix} \qquad Formula 2$$

An amino acid is then used as a vertex of the graph to obtain the protein graph Gₚᵣₒₜₑᵢₙ. As shown in FIG. 4, the protein molecule includes amino acids A, C, D, E, and F. In calculated normalized amino acid distances, d_{AC}, d_{CD}, d_{DE}, and d_{DF} are less than an amino acid distance threshold d₀. Therefore, an adjacency matrix is established based on connection between the amino acid A and the amino acid C, connection between the amino acid C and the amino acid D, connection between the amino acid D and the amino acid E, and connection between the amino acid D and the amino acid F, and then the amino acids involved are used as vertices of a graph, to obtain a protein graph. The protein graph reflects the structural feature of the protein molecule.

For the target molecule, the structural feature of the target molecule may be determined according to an organic structure of the target molecule. For example, FIG. 5 is a schematic flowchart of determining a structural feature of a target molecule according to an embodiment of this disclosure. Steps shown in FIG. 5 may be performed by various electronic devices running the drug screening apparatus, such as a dedicated terminal with a drug screening apparatus, a drug database server, or a server cluster for drug providers. The following describes the steps shown in FIG. 5.

Step 501: Determine an organic structure of a target molecule.

Step 502: Determine atoms and chemical bonds corresponding to the target molecule based on the organic structure of the target molecule.

Step 503: Use the atoms corresponding to the target molecule as nodes in a structural feature of the target molecule.

Step 504: Use the chemical bonds corresponding to the target molecule as edges in the structural feature of the target molecule.

Step 505: Determine the structural feature of the target molecule through the nodes in the structural feature of the target molecule and the edges in the structural feature of the target molecule.

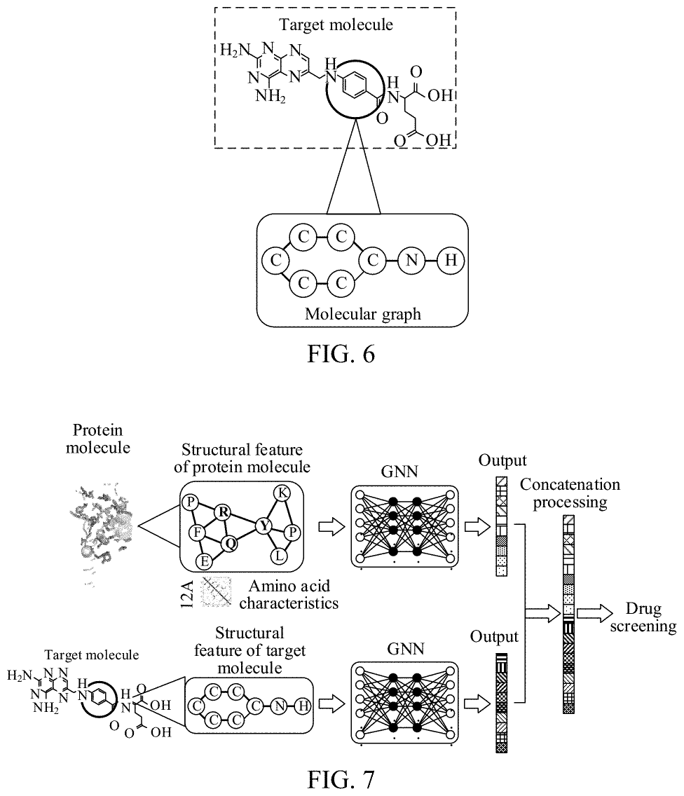

For example, a molecular graph (also referred to as a micromolecular graph) corresponding to the target molecule is determined by using the atoms corresponding to the target molecule as nodes and using the chemical bonds corresponding to the target molecule as edges. The molecular graph reflects the structural feature of the target molecule. For ease of understanding, a schematic diagram of the target molecule and the determined molecular graph shown in FIG. 6 is provided.

After the structural feature of the target molecule is determined, a subsequent step is performed, that is, the protein molecules and the target molecules are screened through a drug screening model.

FIG. 3A includes step 303: Obtain a concatenated node feature corresponding to the protein molecule and the target molecule based on a node information passing sub-network in a drug screening model, the structural feature of the protein molecule, and the structural feature of the target molecule, the node information passing sub-network being a GNN.

For example, an output of the node information passing sub-network in the drug screening model is determined based on the structural feature of the protein molecule and the structural feature of the target molecule. The output is the concatenated node feature corresponding to the protein molecule and the target molecule. The node information passing sub-network is a GNN or may be a part of GNN. GNN is a neural network that directly acts on a graph structure, mainly for processing data with a non-Euclidean spatial structure (graph structure). A GNN may be composed of two modules: a propagation module and an output module. The propagation module is configured to pass information between nodes in the graph and update a state. The output module is configured to define a target function according to different tasks based on vector representation of nodes and edges of the graph. Therefore, by determining all nodes attached to nodes corresponding to a target amino acid chain, information of different amino acid chains in the protein molecule with various structures can be embedded into new nodes that are continuously generated in the GNN to implement embedding representation.

In some embodiments of this disclosure, the obtaining a concatenated node feature corresponding to the protein molecule and the target molecule based on a node information passing sub-network in a drug screening model, the structural feature of the protein molecule, and the structural feature of the target molecule may be implemented in the following manner:

determining a target node feature of a target node based on the structural feature of the protein molecule, the target node being corresponding to an amino acid in the protein molecule; determining an end as an attached edge feature of an edge of the target node based on the structural feature of the protein molecule; determining an attached node feature of an attached node attached to the target node based on the structural feature of the protein molecule; and obtaining, by the node information passing sub-network, the concatenated node feature corresponding to the protein molecule and the target molecule based on the target node feature, the attached edge feature, and the attached node feature.

For example, a target node feature of a target node may be determined based on the structural feature of the protein molecule. A node corresponds to an amino acid in the protein molecule, that is, the target node may correspond to a target amino acid. An end is determined as an attached edge feature of an edge of the target node based on the structural feature of the protein molecule. The end may be determined as the attached edge feature of some edges of the target node, or the end may be determined as the attached edge feature of all edges of the target node. The latter can improve the accuracy and comprehensiveness of the obtained attached edge feature. An attached node feature of an attached node attached to the target node is also determined based on the structural feature of the protein molecule. Similarly, the attached node feature of some attached nodes attached to the target node may be determined, or the attached node feature of all attached nodes attached to the target node may be determined. The node information passing sub-network processes the target node feature, the attached edge feature, and the attached node feature to obtain the concatenated node feature corresponding to the protein molecule and the target molecule.

In the process of determining the output of the node information passing sub-network, a new node corresponding to a target amino acid (for example, a target node embedding representation corresponding to the target amino acid) may be generated through the node information passing sub-network to implement embedding of the amino acid chain into the protein molecule.

In some embodiments, the obtaining, by the node information passing sub-network, the concatenated node feature corresponding to the protein molecule and the target molecule based on the target node feature, the attached edge feature, and the attached node feature may be implemented in the following manner:

generating, by the node information passing sub-network, a target node embedding representation corresponding to the target node based on the target node feature, the attached edge feature, and the attached node feature; obtaining, by the node information passing sub-network, a protein node embedding representation vector corresponding to the protein molecule based on the target node embedding representation; obtaining, by the node information passing sub-network, a target molecule node embedding representation vector corresponding to the target molecule based on the structural feature of the target molecule; and concatenating the protein node embedding representation vector and the target molecule node embedding representation vector to obtain the concatenated node feature corresponding to the protein molecule and the target molecule.

In an embodiment of this disclosure, as shown in FIG. 7, the node information passing sub-network (GNN or a part of GNN) may process the protein molecule and the target molecule respectively. For the protein molecule, the node information passing sub-network processes the target node feature, the attached edge feature, and the attached node feature to generate the target node embedding representation corresponding to the target node, and then may combine (including but not limited to concatenate) node embedding representations corresponding to all nodes respectively (that is, the corresponding target node embedding representation obtained by using each node as the target node), to obtain the protein node embedding representation vector corresponding to the protein molecule. In this case, the embedding representation of the protein molecule is implemented through the node information passing sub-network.

For the target molecule, similarly, the node information passing sub-network may process the structural feature of the target molecule to obtain the target molecule node embedding representation vector corresponding to the target molecule. In this case, the embedding representation of the target molecule is implemented.

Finally, the protein node embedding representation vector and the target molecule node embedding representation vector are concatenated to obtain the concatenated node feature.

In some embodiments, the generating, by the node information passing sub-network, a target node embedding representation corresponding to the target node based on the target node feature, the attached edge feature, and the attached node feature may be implemented in the following manner:

obtaining an initial state feature of the target node based on the target node feature; obtaining an attached node state feature based on the attached node feature of the attached node; combining, by a first information aggregation function of the node information passing sub-network, the attached node state feature and the attached edge feature to obtain a target node information feature; updating, by an update function of the node information passing sub-network, a state feature of the target node based on the initial state feature of the target node and the target node information feature; and generating, by the node information passing sub-network, the target node embedding representation according to the updated attached node state feature.

For example, feature extraction may be further performed on the target node feature to obtain the initial state feature of the target node. Similarly, feature extraction may be further performed on the attached node feature to obtain the attached node state feature. The attached node state feature and the attached edge feature are combined by the first information aggregation function of the node information passing sub-network to obtain the target node information feature. The first information aggregation function may be a concatenation function, that is, the combination may refer to concatenation processing, but not limited thereto. Then, the initial state feature of the target node and the target node information feature are processed through the update function of the node information passing sub-network. The processing results are the updated target node state feature. The update function may be used for linear processing (such as linear transformation operation) and bias processing. Similarly, the node state feature of each node may be updated according to the method of updating the target node state feature. Finally, the updated attached node state feature is processed through the node information passing sub-network to obtain the target node embedding representation of the target node.

In some embodiments, the generating, by the node information passing sub-network, the target node embedding representation according to the updated attached node state feature may be implemented in the following manner:

combining, by a second information aggregation function of the node information passing sub-network, the updated attached node state feature and the attached node feature to obtain a target node embedding feature; and processing, by an activation function of the node information passing sub-network, the target node embedding feature to obtain the target node embedding representation.

For example, the updated attached node state feature and the attached node feature may be combined through the second information aggregation function of the node information passing sub-network to obtain the target node embedding feature. The second information aggregation function may be a concatenation function, that is, the combination may refer to concatenation processing, but not limited thereto. Then, the target node embedding feature is activated through the activation function of the node information passing sub-network to obtain the target node embedding representation.

For ease of understanding, an example in which the node information passing sub-network is a message passing neural network (MPNN) is used for description. The forward propagation of MPNN includes two stages. The first stage is referred to as a message passing stage, and the second stage is referred to as a readout stage. Herein, a graph structure G=(V, E) is given. V may represent a set of nodes v, and E may represent a set of edges e. A plurality of times of message passing process will be performed at the message passing stage. For example, a node v of a specific amino acid in the corresponding protein graph (the structural feature of the protein molecule) may be subjected to a t^{th} message passing with reference to the formula 3 and the formula 4, and an input of each message passing is at least partially derived from an output of a previous message passing:

mᵥ^{t+1}=Σ_{w∈N}_{₍ᵥ₎}Mₜ(hᵥ^{t},h_{w}^{t},e_{wv}) Formula 3

hᵥ^{t+1}=uₜ(hᵥ^{t},mᵥ^{t+1}) Formula 4

For the target node v corresponding to the target amino acid, the node information passing sub-network (that is, the MPNN model F_{node}^{D}(G)) herein may aggregate the attached node state feature h_{w}^{t} of the attached node w attached to the target node v after t times of message passing and the attached edge feature e_{wv} of the edge between each node w and the target node v to generate a new node v through message passing. For example, referring to the formula 5, formula 6, and formula 7, an implementation example is given:

hᵥ⁰=σ(Wᵢₙxᵥ) Formula 5

mᵥ^{d+1}=Σ_{k∈N}_{₍ᵥ₎}cat(hₖ^{d},eᵥₖ) Formula 6

hᵥ^{d+1}=σ(Wₐmᵥ^{d+1}+hᵥ⁰) Formula 7

A person skilled in the art may understand that the formula 5 describes the initial state feature hᵥ⁰ of the target node obtained by performing feature extraction on the initial node information xᵥ (that is, the target node feature) of the target node v. The formula 6 and formula 7 describe the process of each message passing. N(v) is a set of the attached node k attached to the node v, and σ(·) is the activation function of the neural network. Herein, the first information aggregation function uses the concatenation function cat(·) and uses the attached edge feature eᵥₖ of the attached edge between the node v and the attached node k as μ_{attached}, to concatenate with the attached node state feature hₖ^{d} of the adjacency node k after d times of message passing to obtain the target node information feature and mᵥ^{d+1} after d times of message passing. A node update function (corresponding to the update function above) uses the linear transformation operation plus bias operation. After message passing, a new target node state feature hᵥ^{d} of the target node v will be obtained through updating. In some embodiments, the two weights Wᵢₙ and Wₐ are shared during node updating.

After D times of message passing, an additional step of message passing may be performed to calculate the target node embedding representation hᵥ⁰ (corresponding to a new node) as the output of the node information passing sub-network. In some embodiments, this additional step of message passing may be performed in the form of formula 8 and formula 9:

mᵥ⁰=Σ_{k∈N}_{₍ᵥ₎}cat(hₖ^{d},xₖ) Formula 8

hᵥ⁰=σ(W₀mᵥ⁰) Formula 9

A person skilled in the art may understand that, in this embodiment, the formula 8 describes concatenating (performing information aggregation on) the attached node state feature hₖ^{d} of the attached node k after d times of message passing and the attached node feature xₖ of the attached node k to obtain the target node embedding feature mᵥ⁰, and finally the formula 9 describes obtaining the target node embedding representation hᵥ⁰ of the node v according to mᵥ⁰, the output parameter W₀, and the activation function σ(·). The target node embedding representation hᵥ⁰ corresponds to the target amino acid. The target node embedding representation hᵥ⁰ obtained after message passing aggregates the attached node feature of all nodes k attached to the node v and also aggregates the attached edge feature of the edge d between the node k and the node v. In some embodiments, the target node embedding representation hᵥ⁰ corresponding to n nodes v may be used as the output of the node information passing sub-network, that is, the protein node embedding representation vector Hₐ in the following formula is used as the final output of F_{node}^{D}(G):

Hₐ=[h₁^{o}, . . . ,hₙ^{o}] Formula 10

In some embodiments, the concatenating the protein node embedding representation vector and the target molecule node embedding representation vector to obtain the concatenated node feature corresponding to the protein molecule and the target molecule may be implemented in the following manner:

determining a self-attention readout function matching the drug screening model; determining a first node feature vector in the structural feature of the protein molecule and a second node feature vector in the structural feature of the target molecule through the self-attention readout function, the protein node embedding representation vector, and the target molecule node embedding representation vector; and concatenating the first node feature vector and the second node feature vector to obtain the concatenated node feature corresponding to the protein molecule and the target molecule.

For example, if the output H ∈ R^{n*a} of the message passing network (such as the node information passing sub-network) is given, the self-attention weight matrix S (where the self-attention weight matrix matches the self-attention readout function) may be represented by the formula 11:

S=solfmax(W₂ tanh(W₁H^{T})) Formula 11

W₁ ∈ R^{h}^{ₐₜₜₙ}^{×a} and W₂ ∈ R^{r×h}^{ₐₜₜₙ} are learnable parameters. In the foregoing formula, W₁ is a linear transformation, which embeds and transforms n nodes in an a-dimensional space into a hₐₜₜₙ-dimensional space and performs nonlinear mapping through a hyperbolic tangent function tanh(·), then W₂ linearly transforms the embedding in the hₐₜₜₙ-dimensional space into an r-dimensional space to obtain node importance distribution of r different angles, a larger value indicating a more important node, and finally, through the solfmax(·) function, a sum of the importance values of different angles is 1, so that it conforms to the characteristics of a weight distribution.

After the self-attention weight matrix S ∈ R^{r×n} corresponding to n nodes is obtained, a vector representation of a fixed-size graph including node importance may be determined according to the self-attention weight matrix S and the output H of the message passing network:

ξ=flatten(SH),ξ∈R^{r×a} Formula 12

flatten(·) represents that the matrix SH is expanded into a one-dimensional vector.

Further, the protein node embedding representation vector and the target molecule node embedding representation vector may be concatenated, that is, the information of the micro-molecule and the protein may be combined, and the activity after the protein molecule and the target molecule are bound may be predicted based on the concatenated vector representation. In some embodiments, refer to the form of formula 13:

predₐ=FCNₐ(cat(ξₐ^{P},ξₐ^{M})) Formula 13

cat(·) is a concatenation function, FCN is a fully connected neural network, and ξₐ^{P} is the node feature vector representation (that is, the first node feature vector) obtained after Hₐ^{P} obtained by the protein graph (the structural feature of the protein molecule) through the node information passing sub-network and the self-attention readout function are combined. Similarly, ξₐ^{M} is the node feature vector representation (that is, the second node feature vector) obtained after Hₐ^{M} obtained by the micromolecular graph (the structural feature of the target molecule) through the node information passing sub-network and the self-attention readout function are combined, and predₐ represents the concatenated node feature.

Step 304: Predict a first predicted activity value after the protein molecule and the target molecule are bound according to the concatenated node feature.

The predicted activity value of a binding product of the protein molecule and the target molecule may be predicted according to the obtained concatenated node feature. For ease of distinguishing, the obtained predicted activity value herein is named the first predicted activity value. Compared with the solution provided in the related art, which requires a large number of experiments to obtain the first predicted activity value, in the embodiments of this disclosure, the first predicted activity value can be accurately and quickly determined in combination with the GNN, which can greatly reduce labor costs and time costs.

In some embodiments, after step 304, the method further includes: screening molecules in the drug database based on the first predicted activity value.

One first predicted activity value corresponds to one protein molecule and one target molecule, so that the molecules in the drug database may be screened according to the first predicted activity value. In an example, in a case that the protein molecule is fixed, the first predicted activity value obtained by binding the protein molecule and a plurality of target molecules respectively may be determined, and the plurality of target molecules are screened according to the first predicted activity value. For example, the target molecule corresponding to several largest first predicted activity values (such as one largest first predicted activity value) is used as the target molecule screened out, that is, the screening result. In another example, in a case that the target molecule is fixed, the first predicted activity value obtained by binding the target molecule and a plurality of protein molecules respectively may be determined, and the plurality of protein molecules are screened according to the first predicted activity value. For example, the protein molecule corresponding to several largest first predicted activity values (such as one largest first predicted activity value) is used as the protein molecule screened out, that is, the screening result. In another example, the protein molecule and the target molecule may be screened together in a case that there are a plurality of protein molecules and a plurality of target molecules.

FIG. 3B is a schematic flowchart of a drug screening method according to an embodiment of this disclosure. After step 302 shown in FIG. 3A, in step 305 includes the step(s) to obtain a concatenated edge feature corresponding to the protein molecule and the target molecule based on the edge information passing sub-network, the structural feature of the protein molecule, and the structural feature of the target molecule.

In this embodiment of this disclosure, the drug screening model may also include an edge information passing sub-network. Similarly, the edge information passing sub-network may be GNN or a part of GNN.

After the structural feature of the protein molecule and the structural feature of the target molecule are obtained, the structural feature of the protein molecule and the structural feature of the target molecule may be processed through the edge information passing sub-network to obtain the concatenated edge feature corresponding to the protein molecule and the target molecule.

In some embodiments of this disclosure, the obtaining a concatenated edge feature corresponding to the protein molecule and the target molecule based on the edge information passing sub-network, the structural feature of the protein molecule, and the structural feature of the target molecule may be implemented in the following manner:

determining a target edge feature of a target edge based on the structural feature of the protein molecule, the target edge feature being corresponding to two amino acids attached in the protein molecule; determining an adjacent edge feature of an adjacent edge based on the structural feature of the protein molecule, a first-end node of the adjacent edge being corresponding to one of the two amino acids attached, and a second-end node of the adjacent edge being attached to the first-end node; determining an adjacent node feature corresponding to the second-end node; and obtaining, by the edge information passing sub-network, the concatenated edge feature corresponding to the protein molecule and the target molecule based on the target edge feature, the adjacent edge feature, and the adjacent node feature.

For example, the target edge feature of the target edge is determined based on the structural feature of the protein molecule. The edge corresponds to a relationship between two amino acids that satisfies a specific condition, for example, the two amino acids are bound in the protein graph. The target edge may refer to any edge. The adjacent edge feature of the adjacent edge of the target edge is determined based on the structural feature of the protein molecule. The first-end node of the adjacent edge corresponds to one of the two amino acids attached, and the second-end node of the adjacent edge is attached to the first-end node. Herein, the adjacent edge feature of some adjacent edges of the target edge may be determined, or the adjacent edge feature of all adjacent edges of the target edge may be determined.

For the adjacent edge, the adjacent node feature corresponding to the second-end node is also determined. Finally, the target edge feature, the adjacent edge feature, and the adjacent node feature are processed through the edge information passing sub-network to obtain the concatenated edge feature corresponding to the protein molecule and the target molecule.

In some embodiments, the obtaining, by the edge information passing sub-network, the concatenated edge feature corresponding to the protein molecule and the target molecule based on the target edge feature, the adjacent edge feature, and the adjacent node feature may be implemented in the following manner:

generating, by the edge information passing sub-network, an edge embedding representation corresponding to the first-end node based on the target edge feature, the adjacent edge feature, and the adjacent node feature; obtaining, by the edge information passing sub-network, a protein edge embedding representation vector corresponding to the protein molecule based on the edge embedding representation; obtaining, by the edge information passing sub-network, a target molecule edge embedding representation vector corresponding to the target molecule based on the structural feature of the target molecule; and concatenating the protein edge embedding representation vector and the target molecule edge embedding representation vector to obtain the concatenated edge feature corresponding to the protein molecule and the target molecule.

For example, the target edge feature, the adjacent edge feature, and the adjacent node feature are processed through the edge information passing sub-network to obtain the edge embedding representation corresponding to the first-end node. The protein edge embedding representation vector corresponding to the protein molecule is determined based on the edge embedding representation. For example, all edge embedding representations may be combined (such as concatenated) to obtain the protein edge embedding representation vector. In addition, the structural feature of the target molecule is processed through the edge information passing sub-network to obtain the target molecule edge embedding representation vector corresponding to the target molecule.

Finally, the protein edge embedding representation vector and the target molecule edge embedding representation vector are concatenated to obtain the concatenated edge feature corresponding to the protein molecule and the target molecule.

In some embodiments, the generating, by the edge information passing sub-network, an edge embedding representation corresponding to the first-end node based on the target edge feature, the adjacent edge feature, and the adjacent node feature may be implemented in the following manner:

obtaining an initial state feature of the target edge based on the target edge feature; obtaining an adjacent edge state feature based on the adjacent edge feature; combining, by a first information passing function of the edge information passing sub-network, the adjacent edge state feature and the adjacent node feature to obtain a target edge information feature; updating, by an update function of the edge information passing sub-network, a state feature of the target edge based on the target edge information feature and the initial state feature of the target edge; and generating, by the edge information passing sub-network, the edge embedding representation according to the updated adjacent edge state feature.

For example, feature extraction may be further performed on the target edge feature to obtain the initial state feature of the target edge. Feature extraction may be further performed on the adjacent edge feature to obtain the adjacent edge state feature. The adjacent edge state feature and the adjacent node feature are combined through the first information passing function of the edge information passing sub-network to obtain the target edge information feature. Then, the target edge information feature and the initial state feature of the target edge are processed through the update function of the edge information passing sub-network. The processing results are the updated target edge state feature. The update function may be used for linear processing (such as linear transformation operation) and bias processing. Similarly, the edge state feature (such as the adjacent edge state feature) of each edge may be updated according to the method of updating the target edge state feature. Finally, the updated adjacent edge state feature is processed through the edge information passing sub-network to obtain the edge embedding representation.

In some embodiments, the generating, by the edge information passing sub-network, the edge embedding representation according to the updated adjacent edge state feature may be implemented in the following manner:

combining, by a second information passing function of the edge information passing sub-network, the updated adjacent edge state feature and the adjacent node feature to obtain an edge embedding feature corresponding to the first-end node; and processing, by an activation function of the edge information passing sub-network, the edge embedding feature to obtain the edge embedding representation.

For example, the updated adjacent edge state feature and the adjacent node feature may be combined through the second information passing function of the edge information passing sub-network to obtain the edge embedding feature corresponding to the first-end node. The second information passing function may be a concatenation function, that is, the combination may refer to concatenation processing, but not limited thereto. Then, the edge embedding feature is activated through the activation function of the edge information passing sub-network to obtain the edge embedding representation.

For ease of understanding, an example in which the edge information passing sub-network is an MPNN is used for description. The target edge information feature m_{vw}^{d} and the target edge state feature h_{vw}^{d} corresponding to the given target edge feature e_{vw} may be calculated according to the formula 14, formula 15, and formula 16:

h_{vw}⁰=σ(Wᵢₙe_{vw}) Formula 14

m_{vw}^{d+1}=Σ_{k∈N}_{_{(v)\w}}^{Σ}cat(hₖᵥ^{d},xₖ) Formula 15

h_{vw}^{d+1}=σ(W_{b}m_{vw}^{d+1}+h_{vw}⁰) Formula 16

In the formulas 14-16, a person skilled in the art may understand that the adjacent edge set kv corresponding to the target edge feature e_{vw} is a set of all edges with an end being the node v except the edge vw, that is, k ∈ N(v)\w. The formula 14 describes that the initial state feature of the target edge is obtained based on the target edge feature. The information passing function (see formula 15, that is, the first information passing function) herein is similar to the information passing function (see formula 3) in the foregoing node information passing sub-network. The adjacent edge information feature corresponding to each edge vk in the adjacent edge set after d times of message passing and the attached feature μ_{attached} corresponding to each edge vk (that is, the node feature xₖ of the end node k corresponding to each edge vk in the adjacent edge set except the node v, that is, the adjacent node feature) are concatenated. The node update function (see formula 16) herein is also similar to the node update function (see formula 7) in the foregoing node information passing sub-network. The target edge state feature is updated based on the target edge information feature and the initial state feature of the target edge by using the linear transformation operation plus bias operation.

After D rounds of message passing, similarly, the edge information may be transferred into the node information at two ends by aggregating the node information for another round to generate the final target edge embedding representation hᵥ⁰. In some embodiments, this another round of information aggregation may be implemented in the form of formula 17 and formula 18:

mᵥ⁰=Σ_{k∈N}_{₍ᵥ₎}cat(hₖᵥ^{d},xₖ) Formula 17

hᵥ⁰=σ(W₀mᵥ⁰) Formula 18

In this embodiment, a person skilled in the art will understand that the formula 17 describes that the adjacent edge state feature hₖᵥ^{d} of the adjacent edge kv after D times of message passing and the adjacent node feature xₖ of the adjacent node k of another end of the edge kv are concatenated to obtain the edge embedding feature mᵥ⁰; and finally, the formula 18 describes that the edge embedding representation hᵥ⁰ of the node v is obtained according to mᵥ⁰, the output parameter W₀, and the activation function σ(·).

In some embodiments, the edge embedding representation hᵥ⁰ corresponding to n nodes v may be used as the output of the edge information passing sub-network, that is, the edge embedding representation vector H_{b} in the following formula is used as the final output of F_{edge}^{D}(G).

The final output result F_{edge}^{D}(G) herein may be represented as:

H_{b}=[h₁^{o}, . . . ,hₙ^{o}].

In some embodiments, the structural feature of the target molecule may also be processed through the edge information passing sub-network to output the target molecule edge embedding representation vector corresponding to the target molecule.

In some embodiments, the concatenating the protein edge embedding representation vector and the target molecule edge embedding representation vector to obtain the concatenated edge feature corresponding to the protein molecule and the target molecule may be implemented in the following manner:

determining a self-attention readout function matching the drug screening model; determining a first edge feature vector in the structural feature of the protein molecule and a second edge feature vector in the structural feature of the target molecule through the self-attention readout function, the protein edge embedding representation vector, and the target molecule edge embedding representation vector; and concatenating the first edge feature vector and the second edge feature vector to obtain the concatenated edge feature corresponding to the protein molecule and the target molecule.

For example, the self-attention weight matrix S may be obtained according to the formula 11. In some embodiments, in order to enable the feature information extracted by the node information passing sub-network and the edge information passing sub-network to interact during training, the attention parameters W₁ and W₂ in the formula 11 may be shared on the two networks, that is, a set of W₁ and W₂ may be used.

After the self-attention weight matrix S ∈ R^{r×n} corresponding to n nodes is obtained, a vector representation ξ of a fixed-size graph including node importance may be obtained according to the self-attention weight matrix S and the input H from the message passing network.

Further, the protein representation and the target molecule representation may be concatenated, that is, the information of the micro-molecule and the protein may be combined, and the activity after the protein molecule and the target molecule are bound may be predicted based on the concatenated vector representation. In some embodiments, refer to the form of formula 19:

pred_{b}=FCN_{b}(cat(ξ_{b}^{P},ξ_{b}^{M})) Formula 19

cat(·) is a concatenation function, FCN is a fully connected neural network, and ξ_{b}^{P} is the edge feature vector representation (that is, the first edge feature vector) obtained after H_{b}^{P} obtained by the protein graph through the edge information passing sub-network and the self-attention readout function are combined. Similarly, ξ_{b}^{M} is the edge feature vector representation (that is, the second edge feature vector) obtained after H_{b}^{M} obtained by the micromolecular graph through the edge information passing sub-network and the self-attention readout function are combined, and pred_{b} represents the concatenated edge feature.

Step 306: Predict a second predicted activity value after the protein molecule and the target molecule are bound according to the concatenated edge feature.

Herein, the predicted activity value after the protein molecule and the target molecule are bound is predicted according to the concatenated edge feature. For ease of distinguishing, the predicted activity value herein is named the second predicted activity value.

In some embodiments, after step 306, the method further includes: screening molecules in the drug database based on the first predicted activity value and the second predicted activity value.

A person skilled in the art may understand that the predicted activity value of the final drug screening may be at least one of predₐ and pred_{b}, or may be a mean value of predₐ and pred_{b}, or may be a final predicted activity value obtained by calculating predₐ and pred_{b} based on other methods. This is not limited in this disclosure. In addition, the foregoing involved weight parameters in the fully connected FCN may be obtained by training a training set.

In some embodiments, a person skilled in the art may understand that the drug screening model may predict the activity after the target molecule and the protein molecule are bound based only on the node information passing sub-network, for example, predict through the formula 13. Similarly, in some embodiments, the drug screening model may predict the activity after the target molecule and the protein molecule are bound based only on the edge information passing sub-network, for example, predict through the formula 19.

A person skilled in the art may understand that, in the embodiments of this disclosure, what needs to be screened may be a protein molecule, or may be a target molecule, or may be both the protein molecule and the target molecule. This is not limited thereto.

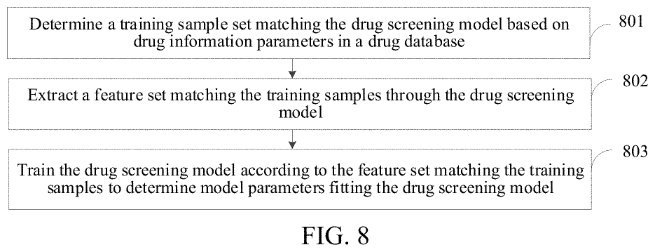

FIG. 8 is a schematic diagram of a process of training a drug screening model according to an embodiment of this disclosure. It may be understood that steps shown in FIG. 8 may be performed by various electronic devices running the drug screening apparatus, such as a dedicated terminal with a drug screening apparatus, a drug database server, or a server cluster for drug providers. The following describes the steps shown in FIG. 8.

Step 801: Determine a training sample set matching the drug screening model based on drug information parameters in a drug database.

The training sample set includes at least one group of training samples. A person skilled in the art may understand that the training sample usually includes a structure of a target molecule and an activity label (also referred to as activity label value) recorded through testing after the target molecule and a specific protein molecule are bound.

Step 802: Extract a feature set matching the training samples through the drug screening model.

Step 803: Train the drug screening model according to the feature set matching the training samples to determine model parameters fitting the drug screening model.

Herein, the trained drug screening model may be configured to perform activity prediction on the bonding of the protein molecule and the target molecule.

In some embodiments, a verification sample set matching the drug screening model may also be determined based on the drug information parameters in the drug database. The verification sample set is configured to train the drug screening model in combination with the training sample set. For example, the verification sample set may be configured to verify whether the drug screening model trained according to the training sample set reaches an expected training effect (such as a set precision, a recall, or an F1 score). If the drug screening model reaches the expected training effect, it is determined that the training is completed; and if the drug screening model does not reach the expected training effect, continue training according to the training sample set.

In some embodiments of this disclosure, the method for training the drug screening model further includes:

determining a multidimensional loss function matching the drug screening model; adjusting parameters (weight parameters) of the drug screening model based on the multidimensional loss function, the adjusted drug screening model being configured to perform activity prediction on the bonding of the protein molecule and the target molecule. In some embodiments, during training of the drug screening model, a plurality of loss functions may be used to perform multi-supervised training on the model. In some embodiments, the loss function may be in the form of dual-branch mean square error (MSE) loss function. For example, the dual-branch MSE loss function may include at least one of formula 20 and formula 21:

L_{pred}_{ₐ}=MSE(predₐ,Target) Formula 20

L_{pred}_{_{b}}=MSE(pred_{b},Target) Formula 21

The formula 20 is used to calculate an MSE between the predicted value predₐ and the activity label of the training sample as a loss value. The formula 21 is used to calculate an MSE between the predicted value pred_{b} and the activity label of the training sample as a loss value. In this case, backpropagation is performed in the drug screening model based on at least one of the two loss values to update trainable parameters in the drug screening model.

In some embodiments of this disclosure, in order to make the two predicted activity values obtained through calculation according to the formula 13 and formula 19 equal, the difference loss of formula 22 may also be included in the loss function, that is, the difference between the two predicted activity values is included in the loss function:

L_{dis}=MSE(predₐ,pred_{b}) Formula 22

Therefore, a certain type of extreme value distribution can be effectively limited, thereby limiting this type of dispersion, effectively improving the robustness of the algorithm to unbalanced data, and effectively preventing the processing result of the drug screening model from overfitting.

In addition, considering that in practical applications, not only can the solution of this disclosure be implemented through a fixed drug screening server, but also the solution of this disclosure can be implemented through a drug screening server group (cluster) due to a large quantity of protein molecules and target molecules in the drug database.

The following further describes an exemplary structure in which the drug screening apparatus provided in the embodiments of this disclosure is implemented as a software module. In some embodiments, as shown in FIG. 2, the software module stored in the drug screening apparatus of the memory 202 may include: an information transmission module 2081, configured to obtain a protein molecule and a target molecule included in a drug database; and an information processing module 2082, configured to: determine a structural feature of a protein molecule and a structural feature of a target molecule; obtain a concatenated node feature corresponding to the protein molecule and the target molecule based on a node information passing sub-network in a drug screening model, the structural feature of the protein molecule, and the structural feature of the target molecule, the node information passing sub-network being a graph neural network (GNN); and predict a first predicted activity value after the protein molecule and the target molecule are bound according to the concatenated node feature.

In some embodiments, the information processing module 2082 is further configured to screen molecules in the drug database based on the first predicted activity value.

In some embodiments, the information processing module 2082 is further configured to determine spatial positions of different amino acid chains in the protein molecule; obtaining a normalized amino acid distance by determine a distance between amino acids in each pair based on the spatial positions of different amino acid chains, and normalize the distance between amino acids in each pair; determine an amino acid matrix diagram corresponding to the protein molecule based on the normalized amino acid distance and an amino acid distance threshold; determine the structural feature of the protein molecule based on the amino acid matrix diagram corresponding to the protein molecule; and determine atoms and chemical bonds corresponding to the target molecule, and determine the structural feature of the target molecule based on the atoms and the chemical bonds corresponding to the target molecule.

In some embodiments, the information processing module 2082 is further configured to determine a target node feature of a target node based on the structural feature of the protein molecule, the target node being corresponding to an amino acid in the protein molecule; determine an end as an attached edge feature of an edge of the target node based on the structural feature of the protein molecule; determine an attached node feature of an attached node attached to the target node based on the structural feature of the protein molecule; and obtain, by the node information passing sub-network, the concatenated node feature corresponding to the protein molecule and the target molecule based on the target node feature, the attached edge feature, and the attached node feature.

In some embodiments, the information processing module 2082 is further configured to generate, by the node information passing sub-network, a target node embedding representation corresponding to the target node based on the target node feature, the attached edge feature, and the attached node feature; obtain, by the node information passing sub-network, a protein node embedding representation vector corresponding to the protein molecule based on the target node embedding representation; obtain, by the node information passing sub-network, a target molecule node embedding representation vector corresponding to the target molecule based on the structural feature of the target molecule; and concatenate the protein node embedding representation vector and the target molecule node embedding representation vector to obtain the concatenated node feature corresponding to the protein molecule and the target molecule.

In some embodiments, the information processing module 2082 is further configured to obtain an initial state feature of the target node based on the target node feature; obtain an attached node state feature based on the attached node feature of the attached node; combine, by a first information aggregation function of the node information passing sub-network, the attached node state feature and the attached edge feature to obtain a target node information feature; update, by an update function of the node information passing sub-network, a state feature of the target node based on the initial state feature of the target node and the target node information feature; and generate, by the node information passing sub-network, the target node embedding representation according to the updated attached node state feature.

In some embodiments, the information processing module 2082 is further configured to combine, by a second information aggregation function of the node information passing sub-network, the updated attached node state feature and the attached node feature to obtain a target node embedding feature; and process, by an activation function of the node information passing sub-network, the target node embedding feature to obtain the target node embedding representation.

In some embodiments, the information processing module 2082 is further configured to determine a self-attention readout function matching the drug screening model; determine a first node feature vector in the structural feature of the protein molecule and a second node feature vector in the structural feature of the target molecule through the self-attention readout function, the protein node embedding representation vector, and the target molecule node embedding representation vector; and concatenate the first node feature vector and the second node feature vector to obtain the concatenated node feature corresponding to the protein molecule and the target molecule.

In some embodiments, the information processing module 2082 is further configured to obtain a concatenated edge feature corresponding to the protein molecule and the target molecule based on the edge information passing sub-network, the structural feature of the protein molecule, and the structural feature of the target molecule; and predict a second predicted activity value after the protein molecule and the target molecule are bound according to the concatenated edge feature.

In some embodiments, the information processing module 2082 is further configured to determine a target edge feature of a target edge based on the structural feature of the protein molecule, the target edge feature being corresponding to two amino acids attached in the protein molecule; determine an adjacent edge feature of an adjacent edge based on the structural feature of the protein molecule, a first-end node of the adjacent edge being corresponding to one of the two amino acids attached, and a second-end node of the adjacent edge being attached to the first-end node; determine an adjacent node feature corresponding to the second-end node; and obtain, by the edge information passing sub-network, the concatenated edge feature corresponding to the protein molecule and the target molecule based on the target edge feature, the adjacent edge feature, and the adjacent node feature.

In some embodiments, the information processing module 2082 is further configured to generate, by the edge information passing sub-network, an edge embedding representation corresponding to the first-end node based on the target edge feature, the adjacent edge feature, and the adjacent node feature; obtain, by the edge information passing sub-network, a protein edge embedding representation vector corresponding to the protein molecule based on the edge embedding representation; obtain, by the edge information passing sub-network, a target molecule edge embedding representation vector corresponding to the target molecule based on the structural feature of the target molecule; and concatenate the protein edge embedding representation vector and the target molecule edge embedding representation vector to obtain the concatenated edge feature corresponding to the protein molecule and the target molecule.

In some embodiments, the information processing module 2082 is further configured to obtain an initial state feature of the target edge based on the target edge feature; obtain an adjacent edge state feature based on the adjacent edge feature; combine, by a first information passing function of the edge information passing sub-network, the adjacent edge state feature and the adjacent node feature to obtain a target edge information feature; update, by an update function of the edge information passing sub-network, a state feature of the target edge based on the target edge information feature and the initial state feature of the target edge; and generate, by the edge information passing sub-network, the edge embedding representation according to the updated adjacent edge state feature.

In some embodiments, the information processing module 2082 is further configured to combine, by a second information passing function of the edge information passing sub-network, the updated adjacent edge state feature and the adjacent node feature to obtain an edge embedding feature corresponding to the first-end node; and process, by an activation function of the edge information passing sub-network, the edge embedding feature to obtain the edge embedding representation.

In some embodiments, the information processing module 2082 is further configured to determine a self-attention readout function matching the drug screening model; determine a first edge feature vector in the structural feature of the protein molecule and a second edge feature vector in the structural feature of the target molecule through the self-attention readout function, the protein edge embedding representation vector, and the target molecule edge embedding representation vector; and concatenate the first edge feature vector and the second edge feature vector to obtain the concatenated edge feature corresponding to the protein molecule and the target molecule.

In some embodiments, the information processing module 2082 is further configured to screen molecules in the drug database based on the first predicted activity value and the second predicted activity value.

In some embodiments, the drug screening apparatus further includes a training module, configured to determine a training sample set matching the drug screening model based on drug information parameters in a drug database, the training sample set including at least one group of training samples; extract a feature set matching the training samples through the drug screening model; and train the drug screening model according to the feature set matching the training samples to determine model parameters fitting the drug screening model.

In some embodiments, the training module is further configured to determine a multidimensional loss function matching the drug screening model; and adjust parameters of the drug screening model based on the multidimensional loss function. The adjusted drug screening model is configured to perform activity prediction on the bonding of the protein molecule and the target molecule.

In some embodiments, in a process of training the drug screening model, a loss function includes at least one of the following: a mean square error (MSE) loss function between the first predicted activity value and an activity label of a training sample; an MSE loss function between the second predicted activity value and the activity label; and an MSE loss function between the first predicted activity value and the second predicted activity value.

The embodiments of this disclosure provide a computer-readable storage medium storing an executable instruction. When the executable instruction is executed by a processor, the processor is caused to perform the drug screening method provided in the embodiments of this disclosure.

In some embodiments, the computer-readable storage medium may be a memory such as an FRAM, a ROM, a PROM, an EPROM, an EEPROM, a flash memory, a magnetic surface memory, an optical disk, or a CD-ROM, or may be any device including one of or any combination of the foregoing memories.

In some embodiments, the executable instructions may be written in any form of programming language (including a compiled or interpreted language, or a declarative or procedural language) by using the form of a program, software, a software module, a script or code, and may be deployed in any form, including being deployed as an independent program or being deployed as a module, a component, a subroutine, or another unit suitable for use in a computing environment.

In an example, the executable instructions may, but do not necessarily, correspond to a file in a file system, and may be stored in a part of a file that saves another program or other data, for example, be stored in one or more scripts in a HyperText Markup Language (HTML) file, stored in a file that is specially used for a program in discussion, or stored in a plurality of collaborative files (for example, be stored in files of one or more modules, subprograms, or code parts).

As an example, the executable instruction may be deployed on one electronic device for execution, or executed on a plurality of electronic devices located at one location, or executed on a plurality of electronic devices distributed at a plurality of locations and interconnected by using a communication network.

The term module (and other similar terms such as unit, submodule, subunit, etc.) in this disclosure may refer to a software module, a hardware module, or a combination thereof. A software module (e.g., computer program) may be developed using a computer programming language. A hardware module may be implemented using processing circuitry and/or memory. Each module can be implemented using one or more processors (or processors and memory). Likewise, a processor (or processors and memory) can be used to implement one or more modules. Moreover, each module can be part of an overall module that includes the functionalities of the module.

Based on the above, compared with conventional drug screening technologies, the embodiments of this disclosure may achieve at least the following technical effects: (1) Through the drug screening model, possible drug-targeted protein interaction pairs can be quickly provided, thereby reducing the cost of drug research and development experiments, speeding up the mining and discovery of new drug functions, reducing the cost if drug screening, and improving the user experience. (2) It can not only effectively express the structural feature of the protein graph and the structural feature of the micromolecular graph through the drug screening model, achieving accurate binding of the protein molecule and the target molecule, but also efficiently process a huge quantity of protein molecules and target molecules included in the drug database, increasing the efficiency of drug screening, and saving the time of drug screening.

The foregoing descriptions are merely preferred embodiments of this disclosure, but are not intended to limit the protection scope of this disclosure. Any modification, equivalent replacement, or improvement made within the spirit and principle of this disclosure shall fall within the protection scope of this disclosure.

What is claimed is:

1. A drug screening method, performed by an electronic device, the method comprising:
obtaining a protein molecule and a target molecule comprised in a drug database;
determining a structural feature of the protein molecule and a structural feature of the target molecule;
obtaining a concatenated node feature corresponding to the protein molecule and the target molecule based on a node information passing sub-network in a drug screening model, the structural feature of the protein molecule, and the structural feature of the target molecule, the node information passing sub-network being a graph neural network (GNN); and
predicting a first predicted activity value after the protein molecule and the target molecule are bound according to the concatenated node feature.

2. The method according to claim 1, further comprising:
screening molecules in the drug database based on the first predicted activity value.

3. The method according to claim 1, wherein determining the structural feature of the protein molecule and the structural feature of the target molecule comprises:
determining spatial positions of different amino acid chains in the protein molecule;
obtaining a normalized amino acid distance by determining a distance between amino acids in each pair based on the spatial positions of different amino acid chains and normalizing the distance between amino acids in each pair;
determining an amino acid matrix diagram corresponding to the protein molecule based on the normalized amino acid distance and an amino acid distance threshold;
determining the structural feature of the protein molecule based on the amino acid matrix diagram corresponding to the protein molecule;
determining atoms and chemical bonds corresponding to the target molecule; and
determining the structural feature of the target molecule based on the atoms and the chemical bonds corresponding to the target molecule.

4. The method according to claim 1, wherein obtaining the concatenated node feature corresponding to the protein molecule and the target molecule comprises:
determining a target node feature of a target node based on the structural feature of the protein molecule, the target node being corresponding to an amino acid in the protein molecule;
determining an end as an attached edge feature of an edge of the target node based on the structural feature of the protein molecule;
determining an attached node feature of an attached node attached to the target node based on the structural feature of the protein molecule; and
obtaining, by the node information passing sub-network, the concatenated node feature corresponding to the protein molecule and the target molecule based on the target node feature, the attached edge feature, and the attached node feature.

5. The method according to claim 4, wherein obtaining, by the node information passing sub-network, the concatenated node feature corresponding to the protein molecule and the target molecule comprises:
generating, by the node information passing sub-network, a target node embedding representation corresponding to the target node based on the target node feature, the attached edge feature, and the attached node feature;
obtaining, by the node information passing sub-network, a protein node embedding representation vector corresponding to the protein molecule based on the target node embedding representation;
obtaining, by the node information passing sub-network, a target molecule node embedding representation vector corresponding to the target molecule based on the structural feature of the target molecule; and
concatenating the protein node embedding representation vector and the target molecule node embedding representation vector to obtain the concatenated node feature corresponding to the protein molecule and the target molecule.

6. The method according to claim 5, wherein generating, the target node embedding representation corresponding to the target node comprises:
obtaining an initial state feature of the target node based on the target node feature;
obtaining an attached node state feature based on the attached node feature of the attached node;
combining, by a first information aggregation function of the node information passing sub-network, the attached node state feature and the attached edge feature to obtain a target node information feature;
updating, by an update function of the node information passing sub-network, a state feature of the target node based on the initial state feature of the target node and the target node information feature; and
generating, by the node information passing sub-network, the target node embedding representation according to the updated attached node state feature.

7. The method according to claim 6, wherein generating the target node embedding representation according to the updated attached node state feature comprises:
combining, by a second information aggregation function of the node information passing sub-network, the updated attached node state feature and the attached node feature to obtain a target node embedding feature; and
processing, by an activation function of the node information passing sub-network, the target node embedding feature to obtain the target node embedding representation.

8. The method according to claim 5, wherein concatenating the protein node embedding representation vector and the target molecule node embedding representation vector to obtain the concatenated node feature corresponding to the protein molecule and the target molecule comprises:
determining a self-attention readout function matching the drug screening model;
determining a first node feature vector in the structural feature of the protein molecule and a second node feature vector in the structural feature of the target molecule through the self-attention readout function, the protein node embedding representation vector, and the target molecule node embedding representation vector; and
concatenating the first node feature vector and the second node feature vector to obtain the concatenated node feature corresponding to the protein molecule and the target molecule.

9. The method according to claim 1, wherein the drug screening model comprises an edge information passing sub-network, and the method further comprises:
obtaining a concatenated edge feature corresponding to the protein molecule and the target molecule based on the edge information passing sub-network, the structural feature of the protein molecule, and the structural feature of the target molecule; and
predicting a second predicted activity value after the protein molecule and the target molecule are bound according to the concatenated edge feature.

10. The method according to claim 9, wherein obtaining the concatenated edge feature corresponding to the protein molecule and the target molecule comprises:
determining a target edge feature of a target edge based on the structural feature of the protein molecule, the target edge feature being corresponding to two amino acids attached in the protein molecule;
determining an adjacent edge feature of an adjacent edge based on the structural feature of the protein molecule, a first-end node of the adjacent edge being corresponding to one of the two amino acids attached, and a second-end node of the adjacent edge being attached to the first-end node;
determining an adjacent node feature corresponding to the second-end node; and
obtaining, by the edge information passing sub-network, the concatenated edge feature corresponding to the protein molecule and the target molecule based on the target edge feature, the adjacent edge feature, and the adjacent node feature.

11. The method according to claim 10, wherein obtaining, by the edge information passing sub-network, the concatenated edge feature corresponding to the protein molecule and the target molecule comprises:
generating, by the edge information passing sub-network, an edge embedding representation corresponding to the first-end node based on the target edge feature, the adjacent edge feature, and the adjacent node feature;
obtaining, by the edge information passing sub-network, a protein edge embedding representation vector corresponding to the protein molecule based on the edge embedding representation;
obtaining, by the edge information passing sub-network, a target molecule edge embedding representation vector corresponding to the target molecule based on the structural feature of the target molecule; and
concatenating the protein edge embedding representation vector and the target molecule edge embedding representation vector to obtain the concatenated edge feature corresponding to the protein molecule and the target molecule.

12. The method according to claim 11, wherein generating, by the edge information passing sub-network, an edge embedding representation corresponding to the first-end node comprises:
obtaining an initial state feature of the target edge based on the target edge feature;
obtaining an adjacent edge state feature based on the adjacent edge feature;
combining, by a first information passing function of the edge information passing sub-network, the adjacent edge state feature and the adjacent node feature to obtain a target edge information feature;
updating, by an update function of the edge information passing sub-network, a state feature of the target edge based on the target edge information feature and the initial state feature of the target edge; and
generating, by the edge information passing sub-network, the edge embedding representation according to the updated adjacent edge state feature.

13. The method according to claim 12, wherein generating, by the edge information passing sub-network, the edge embedding representation according to the updated adjacent edge state feature comprises:
combining, by a second information passing function of the edge information passing sub-network, the updated adjacent edge state feature and the adjacent node feature to obtain an edge embedding feature corresponding to the first-end node; and
processing, by an activation function of the edge information passing sub-network, the edge embedding feature to obtain the edge embedding representation.

14. The method according to claim 11, wherein concatenating the protein edge embedding representation vector and the target molecule edge embedding representation vector to obtain the concatenated edge feature corresponding to the protein molecule and the target molecule comprises:
determining a self-attention readout function matching the drug screening model;
determining a first edge feature vector in the structural feature of the protein molecule and a second edge feature vector in the structural feature of the target molecule through the self-attention readout function, the protein edge embedding representation vector, and the target molecule edge embedding representation vector; and
concatenating the first edge feature vector and the second edge feature vector to obtain the concatenated edge feature corresponding to the protein molecule and the target molecule.

15. The method according to claim 9, further comprising:
screening molecules in the drug database based on the first predicted activity value and the second predicted activity value.

16. The method according to claim 9, wherein in a process of training the drug screening model, a loss function comprises at least one of the following or their combination:
a mean square error (MSE) loss function between the first predicted activity value and an activity label of a training sample;
an MSE loss function between the second predicted activity value and the activity label; or
an MSE loss function between the first predicted activity value and the second predicted activity value.

17. An electronic device, comprising:
a memory, configured to store one or more executable instructions; and
a processor, configured to perform, when executing the one or more executable instructions stored in the memory, steps comprising:
obtaining a protein molecule and a target molecule comprised in a drug database;
determining a structural feature of the protein molecule and a structural feature of the target molecule;
obtaining a concatenated node feature corresponding to the protein molecule and the target molecule based on a node information passing sub-network in a drug screening model, the structural feature of the protein molecule, and the structural feature of the target molecule, the node information passing sub-network being a graph neural network (GNN); and
predicting a first predicted activity value after the protein molecule and the target molecule are bound according to the concatenated node feature.

18. The electronic device of claim 17, wherein the processor is configured to further perform, when executing the one or more executable instructions stored in the memory, step comprising:
screening molecules in the drug database based on the first predicted activity value.

19. The electronic device of claim 17, wherein the processor is configured to determine the structural feature of the protein molecule and the structural feature of the target molecule by:
determining spatial positions of different amino acid chains in the protein molecule;
obtaining a normalized amino acid distance by determining a distance between amino acids in each pair based on the spatial positions of different amino acid chains and normalizing the distance between amino acids in each pair;
determining an amino acid matrix diagram corresponding to the protein molecule based on the normalized amino acid distance and an amino acid distance threshold;
determining the structural feature of the protein molecule based on the amino acid matrix diagram corresponding to the protein molecule;
determining atoms and chemical bonds corresponding to the target molecule; and
determining the structural feature of the target molecule based on the atoms and the chemical bonds corresponding to the target molecule.

20. A non-transitory computer-readable storage medium, storing one or more executable instructions, the one or more executable instructions, when executed by a processor, implementing steps comprising:
obtaining a protein molecule and a target molecule comprised in a drug database;
determining a structural feature of the protein molecule and a structural feature of the target molecule;
obtaining a concatenated node feature corresponding to the protein molecule and the target molecule based on a node information passing sub-network in a drug screening model, the structural feature of the protein molecule, and the structural feature of the target molecule, the node information passing sub-network being a graph neural network (GNN); and
predicting a first predicted activity value after the protein molecule and the target molecule are bound according to the concatenated node feature.
